(19) United States
(12) Patent Application Publication
(10) Pub. No.: US 2023/0346816 A1
(43) Pub. Date: Nov. 2, 2023

(54) COMPOUNDS AND METHODS FOR TREATING VIRAL INFECTION

(71) Applicants: Ameer E. HASSAN, Harlingen, TX (US); Yousef Hasan Ahmad KHALILI, Dubai (AE)

(72) Inventors: Ameer E. HASSAN, Harlingen, TX (US); Yousef Hasan Ahmad KHALILI, Dubai (AE)

(21) Appl. No.: 18/024,399

(22) PCT Filed: Sep. 3, 2021
(86) PCT No.: PCT/US2021/048991

§ 371 Date: Mar. 2, 2023

Related U.S. Application Data
(60) Provisional application No. 63/074,602, filed on Sep. 4, 2020.
(60) Provisional application No. 63/142,669, filed on Jan. 28, 2021.

(51) Int. Cl. A61K 31/7008; A61K 45/06; A61P 31/14

(52) U.S. Cl. CPC A61K 31/7008; A61K 45/06; A61P 31/14

(57) ABSTRACT

The present disclosure relates to compounds and methods for treating symptoms or sequelae resulting from viral infection, including influenza, rhinovirus, or betacoronavirus infection, such as human coronaviruses such as SARS coronaviruses, MERS coronaviruses, and COVID-19, including Acute Respiratory Distress Syndrome (ARDS) associated with the viral infection.

### CROSS-REFERENCE TO RELATED APPLICATIONS

This application claims priority under 35 U.S.C. § 119(e) to U.S. Provisional Application Ser. No. 63/074,602 filed on Sep. 4, 2020 and U.S. Provisional Application Ser. No. 63/142,669 filed on Jan. 28, 2021, the disclosures of both of which are expressly incorporated by reference herein.

### FIELD

The present disclosure relates to compounds and methods for treating symptoms or sequelae resulting from viral infection, including influenza, rhinovirus, or betacoronavirus infection, such as human coronaviruses such as SARS coronaviruses, MERS coronaviruses, and COVID-19, including Acute Respiratory Distress Syndrome (ARDS) associated with the viral infection.

### BACKGROUND

Viral infections represent one of the most prevalent health risks in the human population. Viral infections originate from a variety of viruses including influenza, coronavirus, rhinovirus, norovirus, rotavirus, exanthematous virus, hepatic virus, and the like. The severity of illness resulting from viral infections can range from minimal or mild symptoms to lethal clinical outcomes. Coronaviruses, especially betacoronaviruses, are a group of related RNA viruses that can affect humans and can cause respiratory tract infections that range from mild to lethal. The betacoronavirus that cause human diseases (human coronaviruses, HCoV) include seven members designated as SARS-CoV-1 (SARS), MERS-CoV (MERS), HCoV-HKU1, HCoV-NL63, HCoV-0C43, HCoV-229E, and most recently SARS-CoV-2 (COVID-19). Mild illnesses in humans include some cases of the common cold (which is caused by coronaviruses and is also caused by other viruses, predominantly rhinoviruses), while more lethal varieties can cause SARS, MERS, and COVID-19.

COVID-19 is thought to spread from person to person, mainly through respiratory droplets produced when an infected person breathes, coughs, or sneezes. Emerging data suggests that the severity of COVID-19 may correlate with viral load in the lungs of patients. After inhalation, SARS-CoV-2 infects epithelial cells in the nasal cavity and begins to replicate. Infected cells shed viral particles, which then infect neighboring cells. As the disease progresses, viral particles infect alveolar type II cells in the lung. These cells produce large amounts of viral particles and ultimately die, causing damage to the epithelial lining of the lung. This damage, and the corresponding immunological response, results in a type of pneumonia. As of August 2020, over 25 million people have been infected in at least 200 countries around the world, with most cases being reported in the United States, Brazil, and India, and the worldwide death toll from the virus is quickly approaching 850,000.

The clinical presentation of infection of COVID-19 is primarily manifested as malignant pneumonia. A current list of COVID-19 symptoms identified by the Centers of Disease Control (CDC) include: fever, cough, shortness of breath or difficulty breathing, chills, repeated shaking with chills, muscle pain, headache, sore throat, loss of taste or sense of smell, persistent pain or pressure in the chest, confusion or inability to arouse, bluish lips or face, diarrhea, or vomiting. The severity levels of COVID-19 are generally categorized into three levels: mild illness (generally asymptomatic); severe illness (including measureable breathing difficulties); and critical illness (characterized by respiratory failure, shock, or multi-organ failure). Although the overall mortality rate of COVID-19 is low (1.4-2.3%), patients with comorbidities are more likely to have severe disease and subsequent mortality. Most available studies have shown that diabetes mellitus (DM) is associated with more severe disease, acute respiratory distress syndrome (ARD) and increased mortality.

To date, no effective treatments or preventions of SARS-CoV-2 or the resulting illnesses resulting from SARS-CoV-2 infection have been developed or approved. Thus, there is a need for treatments for viral infections, such as influenza and betacoronaviruses, including SARS-CoV-2, including treatments for the diseases and disorders created or exacerbated by the viral infection.

### SUMMARY

In one aspect, the disclosure provides a method of treating a subject having a viral infection, such as influenza, rhinovirus, or betacoronavirus infection, comprising administering a therapeutically effective amount of N-acetyl glucosamine, or a derivative thereof.

In another aspect, the disclosure provides a pharmaceutical composition comprising N-acetyl glucosamine, or a derivative thereof, and optionally a pharmaceutically acceptable carrier or excipient, wherein the N-acetyl glucosamine, or a derivative thereof, is in a therapeutically effective amount for treating a viral infection, such as influenza, rhinovirus, or betacoronavirus infection.

In another aspect, the disclosure provides a use of N-acetyl glucosamine, or a derivative thereof, in the preparation of a medicament for treating a subject having a viral infection, such as influenza, rhinovirus, or betacoronavirus infection.

Additional embodiments, features, and advantages of the disclosure will be apparent from the following detailed description and through practice of the disclosure. The compounds, methods, and compositions of the present disclosure can be described as embodiments in any of the following enumerated clauses. It will be understood that any of the embodiments described herein can be used in connection with any other embodiments described herein to the extent that the embodiments do not contradict one another.

1. A method of treating a subject having a viral infection, such as influenza, rhinovirus, or betacoronavirus infection, comprising administering a therapeutically effective amount of N-acetyl glucosamine, or a derivative thereof.

2. The method of clause 1, wherein the betacoronavirus infection is SARS-CoV-2 infection.

3. The method of clause 1 or 2, wherein the betacoronavirus infection has caused or exacerbated in the subject one or more of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.

4. The method of any one of clauses 1 to 3, wherein the therapeutically effective amount treats one or more symptoms of the betacoronavirus infection.

5. The method of any of one of clauses 1 to 4, wherein the N-acetyl glucosamine, or a derivative thereof, is administered intravenously, orally, subcutaneously, buccally, transdermally, or nasally.

6. The method of any of one of clauses 1 to 5, wherein the N-acetyl glucosamine, or a derivative thereof, is administered orally.

7. The method of any one of clauses 1 to 6, wherein the therapeutically effective amount of the N-acetyl glucosamine, or a derivative thereof, is in the range of about 200 mg to about 2100 mg.

8. The method of any one of clauses 1 to 7, wherein the therapeutically effective amount of the N-acetyl glucosamine, or a derivative thereof, is administered once a day (QD), twice a day (BID), or three times a day (TID).

9. The method of any one of clauses 1 to 8, wherein the therapeutically effective amount of the N-acetyl glucosamine, or a derivative thereof, is administered twice a day (BID), at a dose of about 300 mg to about 900 mg per dose; or about 600 mg to about 800 mg; or about 700 mg.

10. The method of any of clauses 1 to 9, further comprising administration of one or more additional supplement agents.

11. The method according to clause 10, wherein the one or more additional supplement agents is a vitamin or an essential mineral.

12. The method according to clause 10 or 11, wherein the one or more additional supplement agents is vitamin A, a B vitamin, vitamin C. vitamin D, or zinc.

13. The method of any one of the preceding clauses, wherein the clinical outcome for the patient receiving the treatment is lower rate of ICU admission, reduced hospital length of stay (LOS), lower rate of death, and/or lower rate of hospice initiation.

14. A pharmaceutical composition comprising N-acetyl glucosamine, or a derivative thereof, and optionally a pharmaceutically acceptable carrier or excipient, wherein the N-acetyl glucosamine, or a derivative thereof, is in a therapeutically effective amount for treating a viral infection, such as a betacoronavirus infection.

15. The pharmaceutical composition of clause 14, wherein the N-acetyl glucosamine is in an amount of about 200 mg to about 2100 mg in the composition.

16. The pharmaceutical composition of clause 14 or 15, wherein the N-acetyl glucosamine is in an amount of about 300 mg to about 900 mg; or about 600 mg to about 800 mg; or about 700 mg in the composition.

17. The pharmaceutical composition of any one of clauses 14 to 16, further comprising one or more additional supplement agents.

18. The pharmaceutical composition according to clause 17, wherein the one or more additional supplement agents is a vitamin or an essential mineral.

19. The pharmaceutical composition according to clause 17 or 18, wherein the one or more additional supplement agents is vitamin A, a B vitamin, vitamin C vitamin D, or zinc.

20. Use of N-acetyl glucosamine, or a derivative thereof, in the preparation of a medicament for treating a subject having a viral infection, such as a betacoronavirus infection.

21. The use of clause 20, wherein the betacoronavirus infection is SARS-CoV-2 infection.

22. The use of clause 20 or 21, wherein the betacoronavirus infection has caused or exacerbated in the subject one or more of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), microthrombosis, endotheliopathy and blood clotting disorders leading to thrombosis (i.e. Ischemic Stroke), and elevated blood glucose levels.

23. The use of any one of clauses 20 to 22, wherein the medicament comprises an amount of N-acetyl glucosamine, or a derivative thereof, effective to treat one or more symptoms of the betacoronavirus infection.

24. The use of any of one of clauses 20 to 23, wherein the medicament is administered intravenously, orally, subcutaneously, buccally, transdermally, or nasally.

25. The use of any of one of clauses 20 to 24, wherein the medicament is administered orally.

26. The use of any one of clauses 20 to 25, wherein the medicament comprises an amount of N-acetyl glucosamine, or a derivative thereof, in the range of about 200 mg to about 2100 mg.

27. The use of any one of clauses 20 to 26, wherein the medicament is administered once a day (QD), twice a day (BID), or three times a day (TID).

28. The use of any one of clauses 20 to 27, wherein the medicament is administered twice a day (BID), and the medicament comprises N-acetyl glucosamine, or a derivative thereof, in an amount of about 300 mg to about 900 mg; or about 600 mg to about 800 mg; or about 700 mg in the medicament.

29. The use of any of clauses 20 to 28, wherein the medicament further comprises one or more additional supplement agents.

30. The use according to clause 29, wherein the one or more additional supplement agents is a vitamin or an essential mineral.

31. The use according to clause 29 or 30, wherein the one or more additional supplement agents is vitamin A, a B vitamin, vitamin C. vitamin D, or zinc.

32. The use of any one of clauses 20 to 31, wherein the clinical outcome for the patient receiving the treatment is lower rate of ICU admission, reduced hospital length of stay (LOS), lower rate of death, and/or lower rate of hospice initiation

33. A compound of the formula

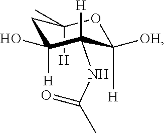

or a pharmaceutically acceptable salt thereof.

### BRIEF DESCRIPTION OF THE DRAWINGS

FIG. 1 is a chart showing the results of a cell proliferation (cytotoxicity) assay of Cpd 1, Cpd 2, and Gln against human chondrocytes at various concentrations with and without the presence of IL-1β.*P<0.05 vs Control (Con). For each compound and concentration, the left bar is without the presence of IL-1β, and the right bar is with the presence of IL-1β.

FIG. 2 is a chart showing the results of an assay for inhibition of NO production by Cpd 1 and Cpd 2 at various concentrations (1, 3.3, and 10 mM), and Gln at 1 and 10 mM. *P<0.05 vs IL-1β.

FIG. 3 is a chart showing the results of an assay for the dose-dependent inhibition of expression of pro-inflammatory gene IL-6 by treatment with Cpd 2 at various concentrations. *P<0.05 vs IL-1β.

FIG. 4 is a chart showing the results of an assay for the dose-dependent inhibition of expression of pro-inflammatory gene COX-2 by treatment with Cpd 2 at various concentrations. *P<0.05 vs IL-1β.

FIG. 5 is a chart showing the results of administration of Cpd 1, Cpd, 2, and Gln on production of IL-6 in IL-113 stimulated human chondrocytes at various concentrations. *P<0.05 vs IL-1β.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination.

### REPRESENTATIVE EMBODIMENTS

In some embodiments, the disclosure provides a method of treating a subject having a betacoronavirus infection comprising administering a therapeutically effective amount of N-acetyl glucosamine, or a derivative thereof. In some embodiments, the disclosure provides the use of N-acetyl glucosamine, or a derivative thereof, in the preparation of a medicament for treating a subject having a betacoronavirus infection. In some embodiments, the disclosure provides a pharmaceutical composition comprising N-acetyl glucosamine, or a derivative thereof, and optionally a pharmaceutically acceptable carrier or excipient, wherein the N-acetyl glucosamine, or a derivative thereof, is in a therapeutically effective amount for treating a betacoronavirus infection. As used herein, the term “N-acetyl glucosamine, or a derivative thereof” (also referred to herein as the compound or compounds) includes 2-(acetylamino)-2-deoxy-0-D-glucopyrano se (N-((2R,3R,4R,5S,6R)-2,4,5-trihydroxy-6-(hydroxymethyl)-tetrahydro-2H-pyran-3-yl)acetamide), represented by the formula

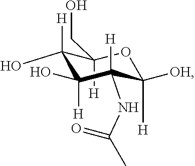

2-(acetylamino)-1,2,3-trideoxy-β-D-glucopyranos e (N-((3R,5S,6R)-5-hydroxy-6-(hydroxymethyl)tetrahydro-2H-pyran-3-yl)acetamide) (a.k.a. Compound 1 or Cpd 1) represented by the formula

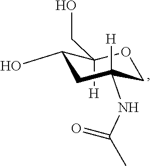

2-(acetylamino)-2,4,6-trideoxy-β-D-glucopyranose (N-((2R,3R,4S,6R)-2,4-dihydroxy-6-methyltetrahydro-2H-pyran-3-yl)acetamide) (a.k.a. Compound 2 or Cpd 2) represented by the formula

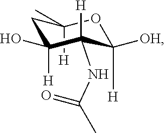

or a pharmaceutically acceptable salt thereof.

In some embodiments, the disclosure provides a compound of the formula

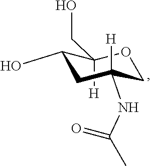

or a pharmaceutically acceptable salt thereof.

In some embodiments, the disclosure provides a compound of the formula

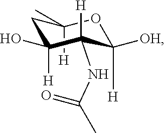

or a pharmaceutically acceptable salt thereof.

In some embodiments, the disclosure provides a composition comprising a compound of the formula

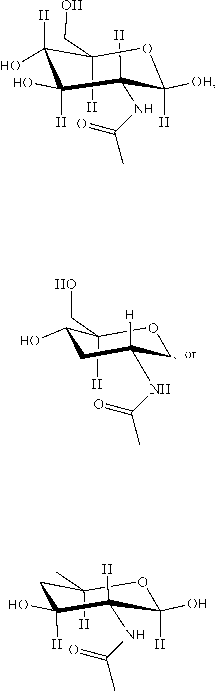

or a pharmaceutically acceptable salt thereof.

As used herein, the term “subject” refers to a human or, in the case of veterinary applications, can be a laboratory, agricultural, domestic, or wild animal. The methods described herein can be applied to subjects including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats.

As used herein, the term “therapeutically effective amount” refers to an amount of a drug or agent that elicits the biological or medicinal response in a subject (i.e. a tissue system, animal or human) that is being sought by a researcher, veterinarian, medical doctor or other clinician, which includes, but is not limited to, alleviation of the symptoms of the disease or disorder being treated. In some embodiments, the therapeutically effective amount is that amount of an active which may treat or alleviate the disease or symptoms of the disease at a reasonable benefit/risk ratio applicable to any medical treatment. In some embodiments, the therapeutically effective amount is that amount of an inactive prodrug which when converted through normal metabolic processes produces an amount of active drug capable of eliciting the biological or medicinal response in a subject that is being sought.

It will be appreciated that the methods, uses, compositions, or compounds described herein can be applied to illnesses resulting from a variety of viral infections, including but not limited to, influenza, coronavirus, and rhinovirus, and the like. In some embodiments, the methods, uses, compositions, or compounds described herein can be applied to illnesses resulting from influenza, rhinovirus, or coronaviruses, especially betacoronaviruses, which can affect humans and can cause respiratory tract infections that range from mild to lethal. In some embodiments, the betacoronavirus include, but are not limited to, SARS-CoV-1 (SARS), MERS-CoV (MERS), HCoV-HKU1, HCoV-NL63, HCoV-0C43, HCoV-229E, SARS-CoV-2 (COVID-19), and the like.

As used herein, COVID-19 refers to coronavirus disease 2019, caused by the SARS-CoV-2 coronavirus. It will be appreciated that populations of pathogenic cells that cause inflammation, for example, as a result of SARS-CoV-2 infection resulting in COVID-19, can lead to a variety of illnesses and symptoms in a subject, such as pneumonia. In some embodiments, the illnesses or symptoms of a subject experiencing influenza, rhinovirus, or coronavirus, especially SARS-CoV-2, infection include, but are not limited to, pneumonia, Acute Respiratory Distress Syndrome (ARDS), systemic inflammatory response syndrome, such as cytokine release syndrome (CRS), a central nervous system disorder, inflammation, multisystem inflammatory syndrome, vasculitis, fever, fever with rigors, fatigue, anorexia, myalgias, arthralgias, nausea, vomiting, headache, rash, kidney disease, intestinal disease, liver disease, diarrhea, tachypnea, hypoxemia, tachycardia, widened pulse pressure, hypotension, increased cardia output, potentially diminished cardiac output, Deep Vein Thrombosis (DVT), microthrombosis, endotheliopathy and blood clotting disorders leading to thrombosis (i.e. Ischemic Stroke), elevated blood glucose levels, elevated D-dimer, hypofibrinogenemia, hypofibrinogenemia with bleeding, azotemia, transaminitis, hyperbilirubinemia, mental state changes, confusion, delirium, word finding difficulty, hallucinations, tremor, dysmetria, altered gait, and seizures. In some embodiments, the illnesses or symptoms of a subject experiencing coronavirus, especially SARS-CoV-2, infection include, but are not limited to, Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), microthrombosis, endotheliopathy and blood clotting disorders leading to thrombosis (i.e. Ischemic Stroke), and elevated blood glucose levels.

In some instances, the illness and/or symptoms experienced by a subject as a result of influenza, rhinovirus, or coronavirus, including SARS-CoV-2, infection can lead to intubation or mechanical ventilation or death. Without being bound by theory, it is believed the methods, uses, compositions, or compounds described herein decreases viral RNA replication by interaction with the glucosamine receptor, which can lead to decreased viral loads, and ultimately leading to a lower incidence of intubation or mechanical ventilation or death.

It will be appreciated that the methods, uses, compositions, or compounds described herein can result in or provide a defined clinical outcome for the patient receiving the treatment, such as lower rate of ICU admission, reduced hospital length of stay (LOS), lower rate of death, lower rate of hospice initiation, reduced intubation rate, reduced mortality rate, and the like. In some embodiments, the clinical outcome for a patient receiving treatment according to the methods and compositions described herein includes lower rate of ICU admission, reduced hospital length of stay (LOS), lower rate of death, and lower rate of hospice initiation.

It will be appreciated that the methods, uses, compositions, or compounds described herein can be administered in any of the modes of administration known in the art. As used herein, “administering” or “administered” includes all means of introducing the compounds and compositions described herein to a subject, including, but are not limited to, oral (po), intravenous (iv), intramuscular (im), subcutaneous (sc), transdermal, inhalation, buccal, ocular, sublingual, nasal, vaginal, rectal, and the like. The methods, uses, compositions, or compounds described herein may be administered in unit dosage forms and/or formulations containing conventional nontoxic pharmaceutically-acceptable carriers, adjuvants, and/or vehicles.

In some embodiments, the methods, uses, compositions, or compounds described herein can be administered orally. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Solid formulations for oral administration may be formulated to be immediate and/or modified release formulations. Modified release formulations include delayed, sustained, pulsed, controlled, targeted and programmed release formulations.

In some embodiments, the methods, uses, compositions, or compounds described herein can be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous means of administration.

In some embodiments, the methods, uses, compositions, or compounds described herein can be co-administered or co-formulated with one or more additional supplemental agents, such as vitamins, essential minerals, drugs, and the like. In some embodiments, the one or more additional supplement agents is vitamin A, a B vitamin, such as folate, vitamin C vitamin D, or zinc.

Any effective regimen for administering the compounds and compositions described herein can be used. For example, compounds and compositions described herein can be administered as single doses, or the doses can be divided and administered as a multiple-dose daily regimen. Further, a staggered regimen, for example, one to five days per week can be used as an alternative to daily treatment. In some embodiments, a subject is administered multiple doses in the methods, uses, compounds, or compositions described herein. In some embodiments, a subjected is administered multiple doses (preferably about 2 up to about 80 doses) with a compound or composition as described herein, for example, at 8-72 hour intervals or at 8-12 hour intervals.

Any suitable course of therapy with the N-acetyl glucosamine, or a derivative thereof, described herein can be used. In one embodiment, individual doses and dosage regimens are selected to provide a total dose administered during a given day of about 200 mg to about 2100 mg; or about 500 mg to about 1500 mg. In some embodiments, the N-acetyl glucosamine, or a derivative thereof, is administered in the methods or uses described herein in a single daily dose (QD), or in a twice daily dose (BID), or a three times daily dose (TID). In some embodiments, the N-acetyl glucosamine, or a derivative thereof, is administered in the methods or uses described herein in a twice daily dose (BID) at a dose of about 300 mg to about 900 mg per dose. In some embodiments, the N-acetyl glucosamine, or a derivative thereof, is administered in the methods or uses described herein in a twice daily dose (BID) at a dose of about 600 mg to about 800 mg. In some embodiments, the N-acetyl glucosamine, or a derivative thereof, is administered in the methods or uses described herein in a twice daily dose (BID) at a dose of about 700 mg. In some embodiments, the N-acetyl glucosamine, or a derivative thereof, is administered in the methods or uses described herein in cycles lasting days a week, 2 weeks, 3 weeks, 4 weeks, 5 weeks, and the like. In some embodiments, the N-acetyl glucosamine, or a derivative thereof, is administered daily in the methods or uses described herein for between 10 and 45 days, or until cessation of treatment is indicated by patient status as observed by a treating physician. In some embodiments, the N-acetyl glucosamine, or a derivative thereof, is administered daily in the methods or uses described herein for between 10 and 20 days, or until cessation of treatment is indicated by patient status as observed by a treating physician. In some embodiments, the N-acetyl glucosamine, or a derivative thereof, is administered daily in the methods or uses described herein for between 25 and 35 days, or until cessation of treatment is indicated by patient status as observed by a treating physician. In some embodiments, the N-acetyl glucosamine, or a derivative thereof, is administered daily in the methods or uses described herein for about 30 days, or until cessation of treatment is indicated by patient status as observed by a treating physician.

It will be appreciated that the unitary daily dosage of the N-acetyl glucosamine, or a derivative thereof, described herein can vary significantly depending on the patient condition, the virus being treated, the route of administration of the N-acetyl glucosamine, or a derivative thereof, and the possibility of co-administration of additional supplemental agents, as described herein. The effective amount to be administered to a patient is based on body surface area, mass, and physician assessment of patient condition.

### Chemical Synthesis

The examples and preparations provided below further illustrate and exemplify particular aspects of embodiments of the disclosure. It is to be understood that the scope of the present disclosure is not limited in any way by the scope of the following examples.

### Example 1: Preparation of 2-(Acetylamino)-1,2,3-Trideoxy-β-D-Glucopyranose (N-((3R,5S,6R)-5-Hydroxy-6-(Hydroxymethyl)Tetrahydro-2H-Pyran-3-Yl)Acetamide)

Compound 1 was prepared according to Scheme 1.

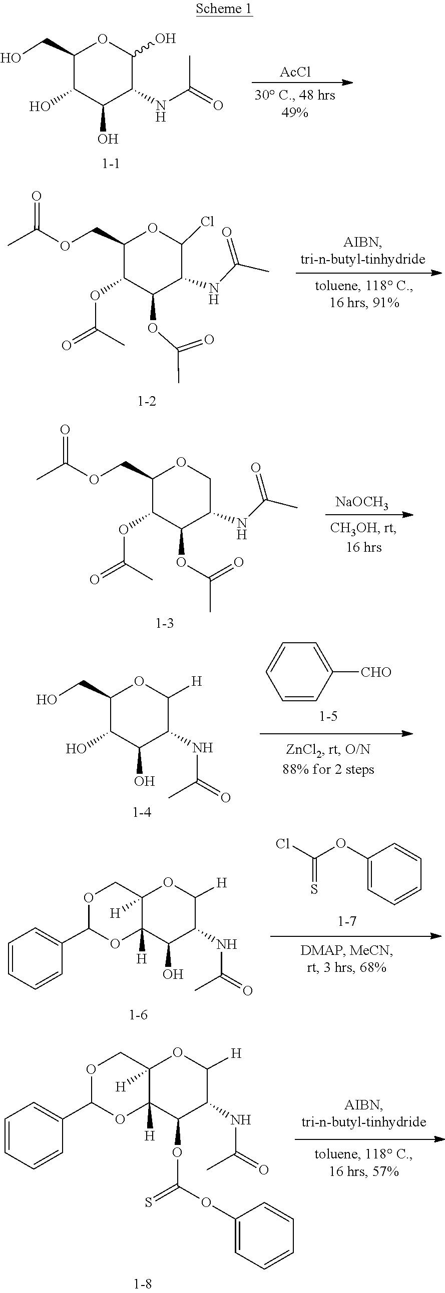

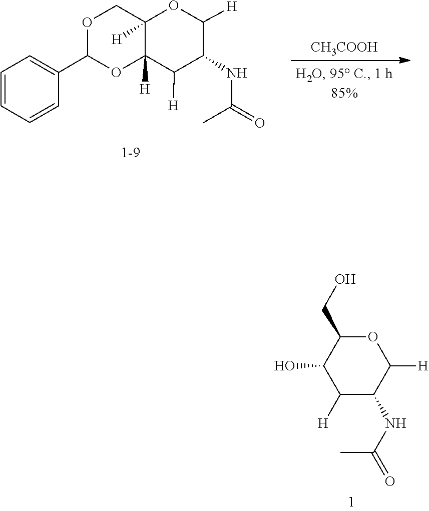

Step 1: Preparation of Compound 1-2

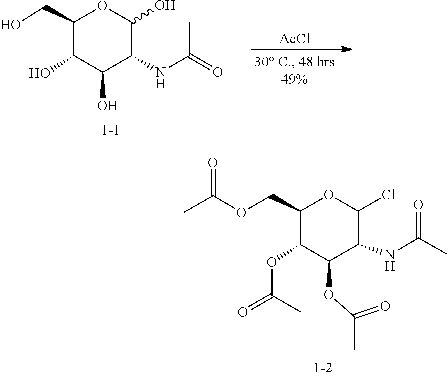 [SMILES: "AcCl" ; CC(=O)N[C@H]1C(O)O[C@H](CO)[C@@H](O)[C@@H]1O ; CC(=O)N[C@H]1C(Cl)O[C@H](COC(C)=O)[C@@H](OC(C)=O)[C@@H]1OC(C)=O]

To a solution of compound 1-1 (200 g) in AcCl (600 mL) was stirred at 30° C. for 48 hrs. The reaction mixture was diluted with DCM (2 L) and poured into ice water (2 L). The mixture was extracted with DCM (1 L×3). The combined organic layers were washed with saturated aqueous NaHCO₃(1 L×2) and brine (800 mL×3). The combined organic layer was dried over anhydrous Na₂SO₄, then filtered and concentrated in vacuo. The residue was purified by column chromatography on silica gel (PE: EA, 1:0 to 1:1) to afford compound 1-2 (162.6 g, 49%) as light yellow solid. TLC: PE: EA=1:2, 12; Rf (compound 1-2)=0.5. LC-MS: 366.25 [M+1]⁺. ¹H NMR (400 MHz, CDCl₃) δ 6.17 (d, J=3.7 Hz, 1H), 5.87 (d, J=8.7 Hz, 1H), 5.31 (dd, J=10.7, 9.4 Hz, 1H), 5.19 (t, J=9.7 Hz, 1H), 4.56-4.47 (m, 1H), 4.31-4.21 (m, 2H), 4.15-4.07 (m, 1H), 2.08 (s, 3H), 2.03 (s, 6H), 1.97 (s, 3H).

Step 2: Preparation of Compound 1-3

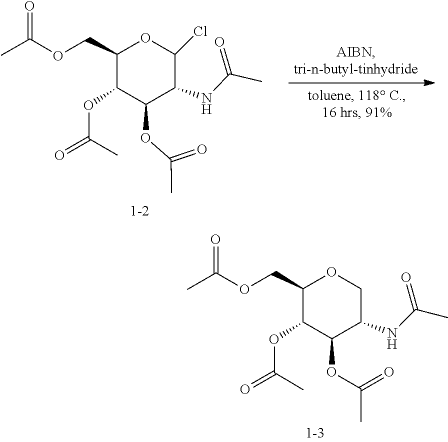 [SMILES: CC(=O)N[C@H]1C(Cl)O[C@H](COC(C)=O)[C@@H](OC(C)=O)[C@@H]1OC(C)=O ; "AIBN,tri-n-butyl-tinhydride" ; CC(=O)N[C@H]1CO[C@H](COC(C)=O)[C@@H](OC(C)=O)[C@@H]1OC(C)=O]

To a suspension of compound 1-2 (162.5 g, 442.3 mmol, 1.0 eq) in toluene (1.6 L) was added AIBN (7.3 g, 44.2 mmol, 0.1 eq) and tri-n-butyltin hydride (193.2 g, 663.5 mmol, 1.5 eq). The reaction mixture was stirred at 118° C. under N₂ for 16 hrs. The reaction mixture was poured into KF aqueous solution (2N, 1.6 L) and stirred for 1 hr. The mixture was filtered, and the filtrate was extracted with DCM (1 L×3). The combined organic layers were washed with brine (900 mL×3). The organic layer was dried with anhydrous Na₂SO₄, filtered and concentrated in vacuo. The residue was purified by column chromatography on silica gel (PE: EA, 3:1 to 0:1) to afford compound 1-3 (134.0 g, 91%) as white solid. TLC: PE: EA=0:1, UV; Rf (compound 1-2)=0.7, Rf (compound 1-3)=0.4. LC-MS: 332.25 [M+1]⁺. ¹H NMR (400 MHz, CDCl₃) δ 5.66 (d, J=7.2 Hz, 1H), 5.07 (t, J=9.5 Hz, 1H), 4.93 (t, J=9.7 Hz, 1H), 4.17 (m, 4H), 3.57-3.49 (m, 1H), 3.15 (m, 1H), 2.10-2.02 (m, 9H), 1.93 (s, 3H).

Step 3: Preparation of Compound 1-4

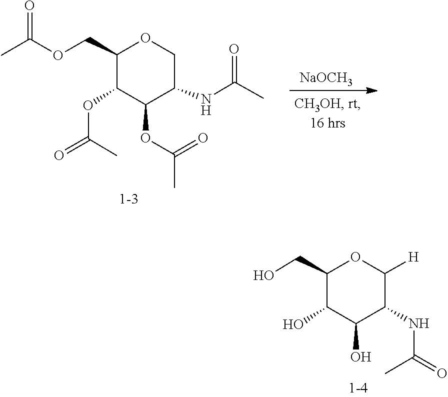

To a solution of compound 1-3 (134.0 g, 404.4 mmol, 1.0 eq) in methanol (1.4 L) was added NaOCH₃ (132 g, 2.42 mol, 6.0 eq). The mixture was stirred at rt for 16 hrs under N₂. The reaction mixture was neutralized. The mixture was filtered and the filtrate was concentrated in vacuo to afford crude compound 1-4 (154 g) as light yellow gum, which was used in next step without further purification. 1H NMR (400 MHz, CD₃OD) δ 3.89 (dd, J=10.9, 5.2 Hz, 1H), 3.84-3.75 (m, 2H), 3.60 (m, 1H), 3.38-3.32 (m, 1H), 3.24 (d, J=8.7 Hz, 1H), 3.14 (m, 1H), 3.09 (t, J=10.9 Hz, 1H), 1.94 (s, 3H).

Step 4: Preparation of Compound 1-6

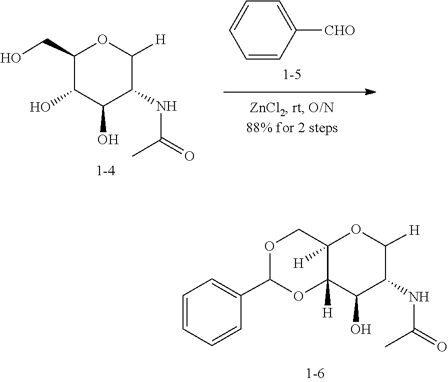

To a suspension of compound 1-4 (98.0 g, 407.6 mmol, 1.0 eq) in benzaldehyde (1-5, 950 mL) was added ZnCl₂ (130.0 g, 955.1 mmol, 2.3 eq). The reaction mixture was stirred at rt under Na for 16 hrs. Water (800 mL) and petroleum ether (PE, 500 mL) was added. The mixture was filtered and the filter cake was washed with PE, and dried to afford compound 1-6 (85.0 g, 88% for 2 steps) as white solid. LC-MS: 294.20 [M+1]⁺. ¹H NMR (400 MHz, CD₃OD) δ 7.53-7.43 (m, 2H), 7.39-7.26 (m, 3H), 5.57 (s, 1H), 4.26-4.18 (m, 1H), 3.97-3.88 (m, 2H), 3.77-3.62 (m, 2H), 3.48 (d, J=9.1 Hz, 1H), 3.33 (d, J=5.0 Hz, 1H), 3.21 (s, 1H), 1.95 (s, 3H).

Step 5: Preparation of Compound 1-8

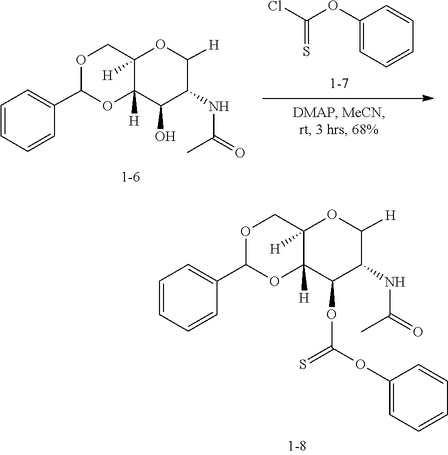

To a suspension of compound 1-6 (102.0 g, 347.7 mmol, 1.0 eq) in acetonitrile (1.8 L) were added DMAP (127.4 g, 1043.1 mmol, 5.0 eq) and compound 1-7 (90.0 g, 521.6 mmol, 1.5 eq). The reaction mixture was stirred at rt under N₂ for 3 hrs. The mixture was filtered. The filtrate was washed with aq HCl (1M, 600 mL×3) and the organic phase was concentrated. The residue was triturated with PE: EA (5:1) to afford compound 1-8 (98.0 g, 68%) as off-white solid. TLC:PE: EA=1:2, UV; Rf (compound 1-6)=0.15, Rf (compound 1-8)=0.6. LC-MS: 430.2 [M+1]⁺, 452.2 [M+Na]⁺. 1H NMR (400 MHz, DMSO-d₆) δ 7.42-7.33 (m, 7H), 7.27 (s, 1H), 7.00 (d, J=7.9 Hz, 2H), 5.68 (s, 1H), 4.26-4.19 (m, 2H), 3.93 (s, 1H), 3.81 (dd, J=11.3, 5.7 Hz, 1H), 3.74 (s, 1H), 3.48 (d, J=11.3 Hz, 2H), 1.80 (s, 3H).

Step 6: Preparation of Compound 1-9

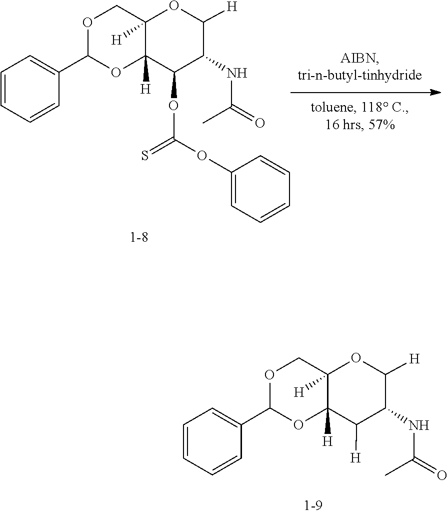

To a suspension of compound 1-8 (78.0 g, 181.7 mmol, 1.0 eq) in toluene (780 mL) was added AIBN (9.0 g, 54.5 mmol, 0.3 eq) and tri-n-butyltin hydride (264.6 g, 908.5 mmol, 5.0 eq). The reaction mixture was stirred at 118° C. under N₂ for 16 hrs. The reaction mixture was poured into aqueous KF solution (2N, 1.6 L) and the mixture was stirred for 1 hr. Then the mixture was filtered and the filtrate was extracted with DCM (800 mL×3). The combined organic layers were washed with brine (900 mL×3), and concentrated in vacuo. The residue was purified by column chromatography on silica gel (PE: EA, 3:1 to 0:1) to afford compound 1-9 (28.8 g, 57%) as white solid. TLC: PE: EA=1:2, 12; Rf (compound 1-8)=0.5, Rf (compound 1-9)=0.3. LC-MS: 278.25 [M+1]⁺. ¹H NMR (400 MHz, CD₃OD) δ 7.43 (m, 2H), 7.32 (m, 3H), 5.58 (s, 1H), 4.20 (m, 1H), 4.05 (m, 1H), 3.98-3.91 (m, 1H), 3.68 (t, J=10.4 Hz, 1H), 3.63 (d, J=1.8 Hz, 1H), 3.26-3.19 (m, 1H), 3.12 (t, J=10.8 Hz, 1H), 2.30-2.22 (m, 1H), 1.91 (s, 3H), 1.58 (q, J=11.6 Hz, 1H).

Step 7: Preparation of Compound 1

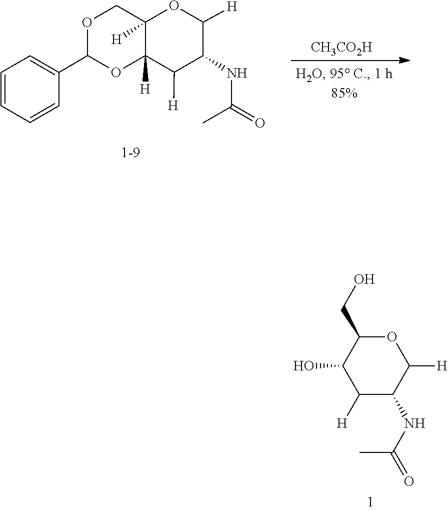

To a suspension of compound 1-9 (12.0 g, 42.3 mmol, 1.0 eq) in acetic acid (150 mL) was added water (300 mL). The reaction mixture was stirred at 95° C. under Na for 1 hr. The reaction mixture was concentrated under reduced pressure. The residue was dissolved in toluene (100 mL) and the mixture was washed with water (30 mL). The organic phase was concentrated and the residue was purified by column chromatography on silica gel (DCM: MeOH, 50:1 to 15:1) to afford Compound 1 (7.0 g, 85%) as white solid. LC-MS: 190.20 [M+1]⁺. ¹H NMR (400 MHz, CD₃OD) δ 3.94-3.79 (m, 3H), 3.61-3.52 (m, 1H), 3.48-3.41 (m, 1H), 3.05-2.95 (m, 2H), 2.25-2.18 (m, 1H), 1.90 (s, 3H), 1.37 (q, J=11.7 Hz, 1H).

### Example 2: Preparation of 2-(Acetylamino)-2,4,6-Trideoxy-β-D-Glucopyranose (N-((2R,3R,4S,6R)-2,4-Dihydroxy-6-Methyltetrahydro-2H-Pyran-3-Yl)Acetamide)

Compound 1 was prepared according to Scheme 2.

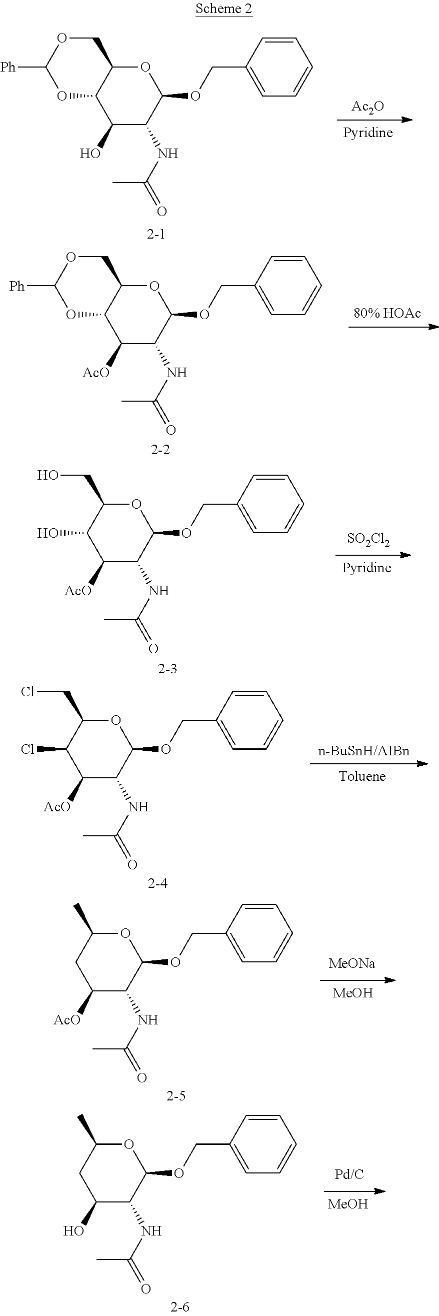

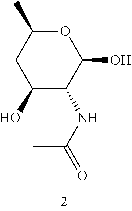

Step 1: Preparation of Compound 2-2

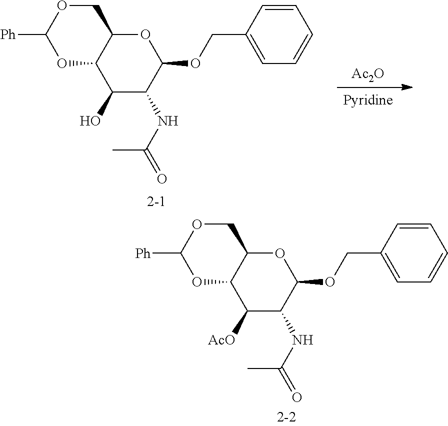

To a solution of compound 2-1 (59 g, 147.7 mmol) in pyridine (413 mL) was added acetic anhydride (60 mL) at room temperature (25-30° C.). After addition, the resulting mixture was stirred at room temperature (25-30° C.) overnight. The reaction was monitored by TLC. After compound 2-1 was consumed completely, the reaction mixture was concentrated under reduced pressure at 50° C. The residue was co-concentrated under reduced pressure with toluene (300 mL×4) to afford compound 2-2 (64 g, 98%) as a white solid.

Step 2: Preparation of Compound 2-3

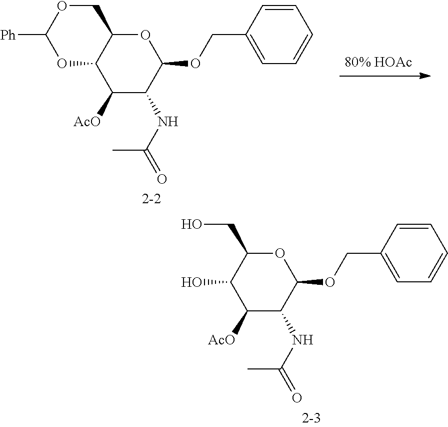

A solution of compound 2-2 (64 g) in 80% HOAc (1.9 L HOAc in 500 mL water) was heated and stirred at 65˜70° C. for 3 hours. The reaction was monitored by LC-MS. After compound 2-2 was consumed completely, the reaction mixture was concentrated under reduced pressure at 50° C. The residue was co-concentrated under reduced pressure with toluene (300 mL×3) to afford compound 2-3 (58.8 g) as a solid.

Step 3: Preparation of Compound 2-4

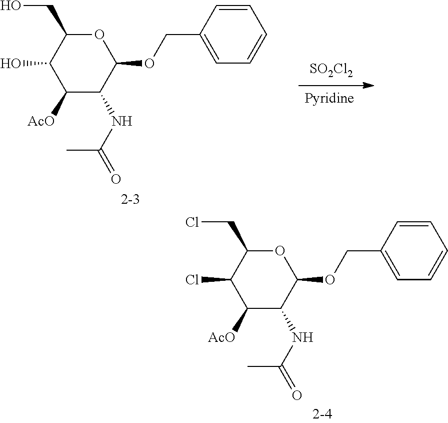

To a solution of compound 2-3 (58.8 g, 166.4 mmol) in pyridine (420 mL) under N₂ atmosphere at −10° C., was added sulfuryl chloride (56.2 g, 416 mmol) dropwise below 0° C. The reaction mixture was stirred at 0° C. for 30 minutes, then warmed up to room temperature (25˜30° C.) and stirred for another 3 hours. The reaction was monitored by LC-MS. After compound 2-3 was consumed completely, the reaction mixture was concentrated under reduced pressure at 50° C., then toluene (300 mL) was added to the residue and co-concentrated to remove pyridine. To the residue was added chloroform (500 mL), water (350 mL) and chloroform (300 mL). After separation, the organic layer was washed with water (300 mL×3), dried over anhydrous Na₂SO₄ and concentrated. Toluene (200 mL) was added and co-concentrated. The residue was triturated with PE/EA (10/1, 420 mL) at room temperature for 4 hours to afford compound 2-4 (39.8 g) as a yellow solid.

Step 4: Preparation of Compound 2-5

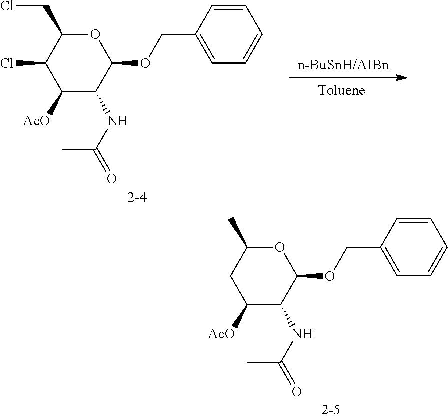 [SMILES: CC(=O)N[C@H]1[C@H](OCc2ccccc2)O[C@H](CCl)[C@H](Cl)[C@@H]1OC(C)=O ; "n-BuSnH/AIBn" ; CC(=O)N[C@H]1[C@H](OCc2ccccc2)O[C@H](C)C[C@@H]1OC(C)=O]

To a solution of compound 2-4 (39.8 g, 101.98 mmol) in toluene (800 mL) under N₂ atmosphere was added n-Bu₃SnH (59.2 g, 204.1 mmol) and AIBN (8.4 g, 51.2 mmol). The mixture was heated to 110° C. (inner temperature) and stirred for 8 hours.

The reaction was monitored by LCMS. Mono-Cl intermediate remained by LCMS (m/z: 356), then n-Bu₃SnH (0.5 eq) and AIBN (0.1 eq) were added every 5 hours until no mono-Cl intermediate remained by LCMS. LCMS also showed small amount of de-acetyl product (compound 2-6).

The reaction mixture was concentrated at 50° C., then co-concentrated with petroleum ether (100 mL×3). The residue was triturated with petroleum ether (200 mL) at −30° C. for 1 hour and viscous solid formed. The supernatant was collected and the viscous solid re-triturated with petroleum ether (200 mL) at −30° C. for 1 hour. The trituration process repeated 3-4 times until sandy solid obtained. The solid was filtered to afford compound 5 (40 g), meanwhile, the combined supernatant was purified by flash column (PE/EA=5/11/1) to afford another batch of compound 2-5 (2 g) (100%).

Step 5: Preparation of Compound 2-6

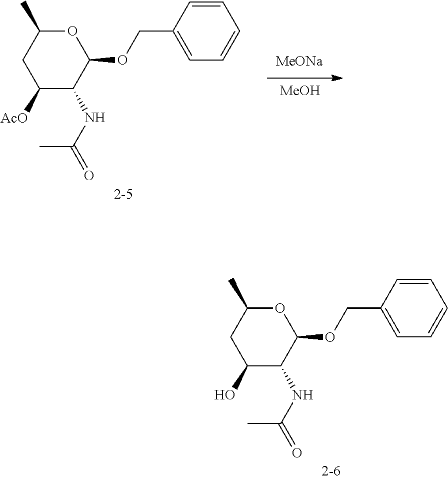 [SMILES: "MeONa" ; "MeOH" ; CC(=O)N[C@H]1[C@H](OCc2ccccc2)O[C@H](C)C[C@@H]1OC(C)=O ; CC(=O)N[C@H]1[C@H](OCc2ccccc2)O[C@H](C)C[C@@H]1O]

To a 2 L three-neck flask was charged dry methanol (820 mL) and cooled to 0° C. under N₂ atmosphere, then sodium methanolate (11.1 g, 205.6 mmol) was added and stirred until clear solution formed. Compound 2-5 (32.8 g, 102.1 mmol, theoretical quantity) dissolved in dry methanol (100 mL) was added into above solution dropwise at 0° C. After addition, the mixture was warmed up to room temperature (25-30° C.) and stirred for 2 hours. The reaction was monitored by LC-MS. After compound 2-5 was consumed completely, the mixture was concentrated under reduced pressure at 50° C. Water (100 mL) was added, extracted with ethyl acetate (100 mL×3). The organic layers were dried over anhydrous Na₂SO₄ and concentrated. The crude was purified by flash column (PE/EA, 5/1-2/1-1/3) to afford compound 2-6 (19 g, 67%).

Step 6: Preparation of Compound 2

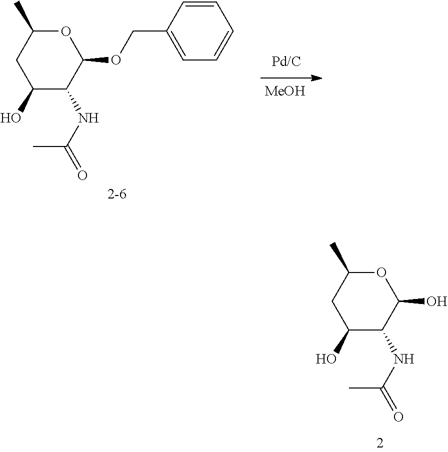 [SMILES: "MeOH" ; CC(=O)N[C@H]1[C@H](OCc2ccccc2)O[C@H](C)C[C@@H]1O ; "Pd/C" ; CC(=O)N[C@H]1[C@H](O)O[C@H](C)C[C@@H]1O]

To a solution of compound 2-6 (8 g, 28.64 mmol) in methanol (800 mL) was added 10% Pd/C (40 g, 50% water content). The reaction was stirred at room temperature (25-30° C.) overnight. The reaction mixture was monitored by LC-MS. After compound 2-6 was consumed completely, the mixture was filtered through a pad of Celite, and washed with MeOH/H₂O (1/1, 400 mL×3). The filtrate was concentrated and re-dissolved in methanol (100 mL), then filtrated through Celite. The filtrate was concentrated and co-concentrated with methanol (50 mL×3) to afford 5 g white solid. The solid was triturated with ethyl acetate (50 mL) to afford compound 2 (4.0 g, 74%). LCMS: 190.20 [M+H]+. 1H NMR (400 MHz, DMSO-d₆): δ7.55 (s, 1H), 6.24 (s, 1H), 4.89 (s, 1H), 4.54 (s, 1H), 3.98-3.95 (m, 1H), 3.66-3.62 (m, 1H), 3.45-3.40 (m, 1H), 1.87-1.83 (m, 1H), 1.79 (s, 3H), 1.13-1.07 (m, 1H) 1.03 (s, 3H).

### Biological Examples

### In Vitro Examples

Materials and Data Analysis:

Isolation and culture of primary human chondrocytes:

Normal cartilage were obtained from autopsy services at the hospital of University of Virginia, approved by the Institutional Review Board. Articular cartilage were harvested from the femoral condyles and the tibial plateaus. Cartilage shavings were harvested at the operation room and placed in tissue culture medium (DMEM, 10% FBS, penicillin, streptomycin), and shipped to the laboratory at 4° C. Chondrocytes were isolated by incubation of the cartilage fragments with 0.01% collagenase NB4 (Fisher Scientific, Catalog No. 50-204-1257) for 4 hrs at 37° C. Cells were maintained in continuous monolayer cultures in DMEM containing 10% FBS. Experiments were performed with cells less than 3 passages. When necessary, trypsin were used for cell passage.

Data Analysis:

Each experiment was duplicated. All groups in each assay were triplicated and the obtained values were presented as means±SD and analyzed using one-way ANOVA followed by the Bonferroni/Dunnet's test. The level of statistical significance between two groups was set at P<0.05.

### Example 3: Cytotoxic Assay

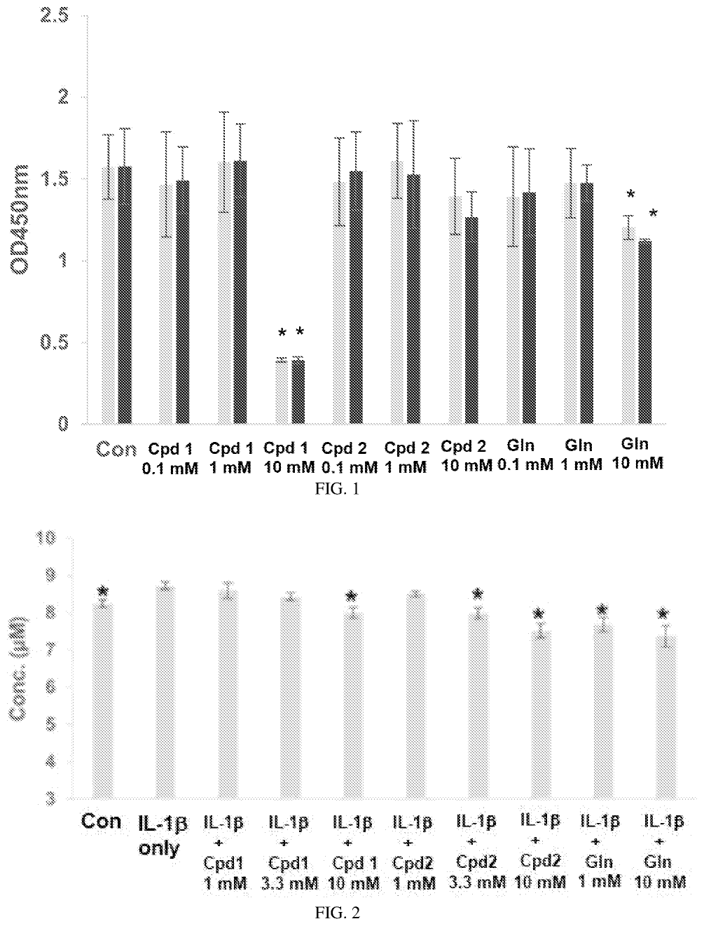

Cells, as prepared in the MATERIALS section, were seeded in a 96-well plate (6×10⁴/well) and incubated with culture medium overnight. Cells were then treated with Compound 1, Compound 2, or Glucosamine (Gln) at various concentrations in DMEM containing 2% FBS (0.1, 1, and 10 mM) for 24 hrs. The WST-1 reagent (Fisher Scientific, Catalog No. 50-100-3295) were used for cell toxic assays, following the instructions provided by the manufacturer. The results are shown in FIG. 1.

### Example 4: Detection of Nitric Oxide Production

Cells, as prepared in the MATERIALS section, were seeded in a 96-well plate (6×10⁴/well) and incubated with culture medium overnight. Cells were then treated with Compound 1 or Compound 2 at various concentrations in DMEM containing 2% FBS (1, 3.3, and 10 mM) for 24 hrs, or Glucosamine (Gln) at various concentrations in DMEM containing 2% PBS (1 or 10 mM) for 24 hrs. The supernatants were harvested for nitrite assay by using the Griess reagent system (Fisher Scientific, Catalog No. G7921). The optical density (OD) were determined at 530 nm on a microplate reader within 30 minutes. The results are shown in FIG. 2.

### Example 5: Gene Expression Analysis

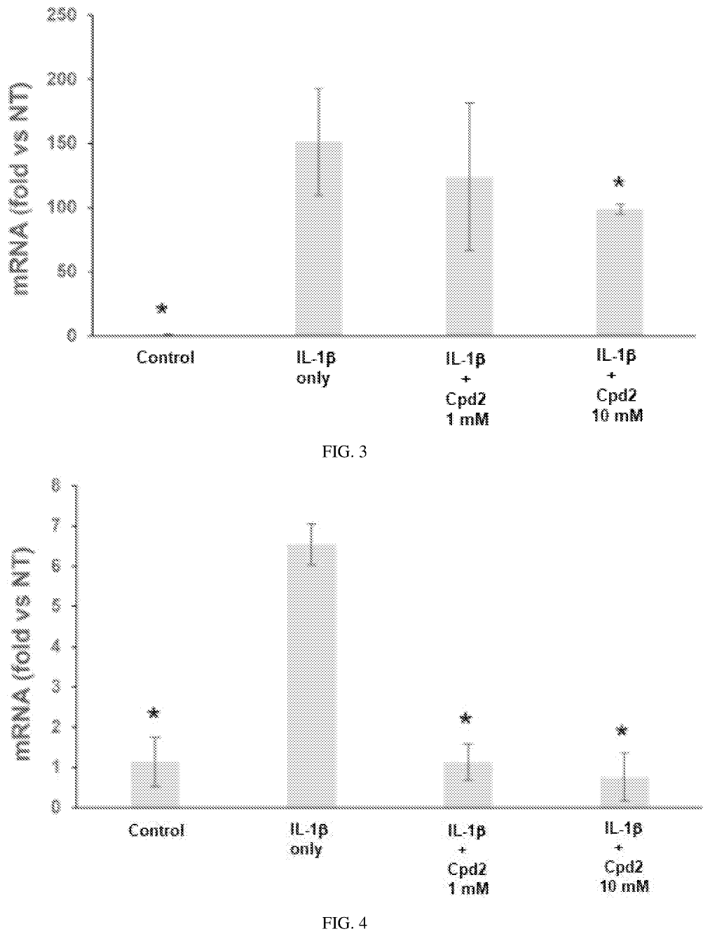

Cells, as prepared in the MATERIALS section, were seeded in a 24-well plate (3×10⁵/well) and incubated with culture medium overnight. Cells were then treated with Compound 2 at various concentrations in DMEM containing 2% FBS (1 or 10 mM) for 24 hrs. Total RNA were extracted and purified from cells using an RNeasy kit (QIAGEN Sciences, Valencia, CA) according to the protocol provided by the manufacturer. Synthesis of cDNA from total RNA and the quantitative PCR were carried out by using the iscript™ cDNA synthesis kit and the iQ™ SYBR Green Supermix kit (Bio-Rad Laboratories, Hercules, CA), respectively. The target genes included COX-2 and IL-6. Gene of 18s ribosomal RNA were used as an internal control. The threshold cycle (CT) value were calculated from amplification plots. Data were analyzed using the 2-ΔΔCT method with 18s rRNA serving as the reference. Gene expression were normalized to the control group (NT) in each experiment and represented as fold of change. The results are shown in FIGS. 3 and 4.

### Example 6: Detection of IL-6 Level

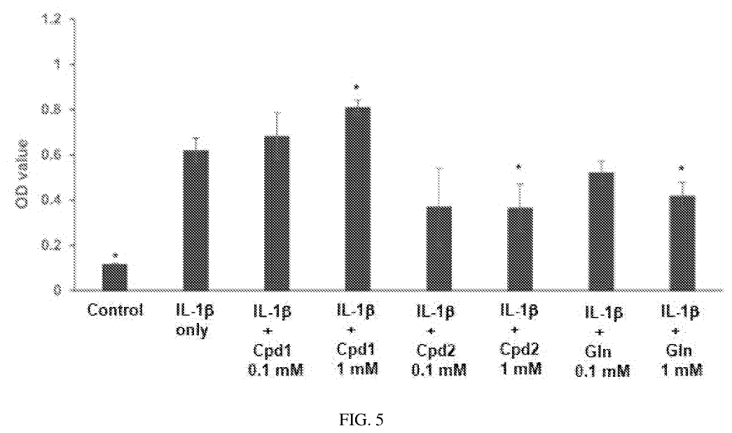

Cells, as prepared in the MATERIALS section, were seeded in a 96-well plate (6×10⁴/well) and incubated with culture medium overnight. Cells were then treated with Compound 1, Compound 2, or Glucosamine (Gln) at various concentrations (0.1 or 1 mM) in DMEM containing 2% FBS for 24 hrs. The supernatants were harvested for assaying production of IL-6 with the Invitrogen™ IL-6 Human ELISA Kit (Fisher Scientific, Catalog No. 5018008), following the instructions provided by the manufacturer. The resultant solutions were read at 450 nm on a microplate reader. The results are shown in FIG. 5.

### In Vivo Examples

### Example 7

To assess the use of N-acetyl glucosamine, a single-center, prospective, observational cohort study was carried out in adult patients presenting to the emergency department of Valley Baptist Medical Center (Harlingen, TX, USA) with COVID-19 symptoms. Consecutive patients were immediately administered 700 mg NAG every 12 hours as first-line treatment upon admission. Patients who subsequently tested positive for COVID-19 through reverse transcription polymerase chain reaction (RT-PCR) were consented and enrolled in the study; those who tested negative for COVID-19 were not included in this study. In addition to NAG, patients in the treatment group received standard of care at the discretion of the attending physician, including antibiotics, antivirals, corticosteroids, and convalescent plasma. Patients continued to receive NAG and were followed until study exit, which occurred at expiration, discharge, or 30 days.

Inclusion criteria, which remained unchanged for the duration of the study, stipulated that all patients had to be >18 years old; receive NAG as first-line treatment; present with shortness of breath, and optionally present with other COVID-19 symptoms (including fever, cough, sore throat, nasal congestion, malaise, headache, muscle pain, loss of taste and/or smell, diarrhea, and vomiting); clinical diagnosis of COVID-19 by RT-PCR; hospital admittance due to COVID-19; and no intubation prior to hospitalization and enrollment in the current study. Patients were excluded if they were <18 years old upon admission, had an allergy to NAG or shellfish, currently taking warfarin, or currently pregnant or lactating.

Upon admission, the research team recorded patient demographics, comorbidities, symptoms, disease severity (as assessed by the World Health Organization [WHO] Ordinal Scale for Clinical Improvement; Table 1), need for supplemental oxygen, and time from symptom onset until hospital arrival. The research team also collected bloodwork for the following at admission: white blood cell count (WBC), hematocrit (HCT), hemoglobin (HBG), C-reactive protein (CRP), procalcitonin (PCT), interleukin-6 (IL-6), and erythrocyte sedimentation rate (ESR). During the study period, discretionary treatments and interventions were recorded daily until study exit.

TABLE 1

| | | Scale |
|---|---|---|
| Illness Severity | Description | Score |
| Not infected | No symptoms or clinical evidence of | 0 |
| | infection | |
| Mild illness | Very few or mild symptoms | 1 |
| | Symptoms managed adequately at home | 2 |
| Moderate illness | Hospitalized, no supplemental oxygen use | 3 |
| | Hospitalized, supplemental oxygen use | 4 |
| Severe illness | Non-invasive breathing support (ventilation, | 5 |
| | high-flow oxygen) | |
| | Invasive breathing support (intubation, | 6 |
| | mechanical ventilation) | |
| | Life-saving ventilation and organ support | 7 |
| Death | Death | 8 |

The primary outcomes of interest were rate of intubation, hospital LOS, and mortality following rapid administration of 700 mg NAG for COVID-19-related symptoms. Secondary outcomes of interest included intensive care unit (ICU) admission, ICU LOS, supplemental oxygen duration, rate of hospice initiation, and poor clinical outcome (defined as combined death/hospice initiation).

Comparison Groups and Statistical Analysis

Beginning on the study start date, the previous 100 COVID-19-positive consecutive patients admitted to Valley Baptist Medical Center were retrospectively identified via chart review to serve as the control arm. Data for these patients was collected before commencement of the NAG trial. Univariate analysis was first performed for all primary and secondary outcomes, followed by multivariate analysis for primary outcomes and select secondary outcomes that approached significance and had sufficient frequency of occurrence to be meaningful. Due to its status as a phase I pilot study and the urgency of releasing information about COVID-19, a priori sample size calculations were not performed for this study; instead, samples were based on cost, patient availability, limited timeline, and best practices in the view of the principal investigator.

Continuous parameters were assessed for normality based on cross-validation using Anderson-Darling, D'Agostino-Pearson omnibus, Shapiro-Wilk, and Kolmogorov-Smirnov tests. Comparisons of normally distributed data between groups were analyzed using unpaired Student's t-tests. Effect sizes from t-tests were reported as mean differences with 95% confidence intervals (CIs) based on normal approximation. Comparisons of nonparametric data between groups were analyzed using the Mann-Whitney U test. Effect sizes from nonparametric tests were reported as Hodge's-Lehman differences (H-L Diff) and their respective 95% CIs. Comparisons of dichotomous data between groups were analyzed using Fisher's exact binomial test. Effect sizes from Fisher's exact test were reported as odds ratios (ORs) with 95% Cis computed using the Baptista-Pike method. A correlation matrix showing the strength and direction of correlation between covariates and outcomes was generated using Spearman's rank correlation test via the ‘corrplot’ package in R. Spearman's rank correlation was used to provide a more robust measurement of correlation in the face of high leverage outliers. Simple linear or logistic regressions were used to evaluate potential predictors of primary and secondary outcomes. Multivariate regression using best subset selection was performed based on model comparison using adjusted R² values (linear regressions) or adjusted pseudo-R² values (logistic regressions). Model predictive performance of multiple linear regression was also evaluated by root mean square error (RMSE) values and multiple logistic regressions were evaluated by area under the receiver operating characteristic curve (ROC). Comparisons of best subset selection with models including the full set of covariates considered for multivariate analysis was also performed. Best subset selection was carried out using the ‘leaps’ package in R. P-values ≤0.05 were considered significant for all analyses. All statistics were performed in RStudio (Version 1.3.959, RStudio, PBC, Boston, MA).

Of the 50 patients enrolled in the prospective cohort study, 48 patients had available follow-up data. The treatment group had median age of 63 years (range: 29-88) and was 50.0% (24/48) male, whereas the patients in the control arm had median age of 68 years (range: 23-95) and was 62.0% (62/100) male. The NAG group was significantly younger than the control group (H-L Diff: 5.0 [95% CI: 0.0; 10.0], p=0.049). Time from symptom onset to admission was significantly longer in the NAG group compared to the control group (H-L Diff: −3.0 [95% CI: −6.0; −1.0], p=0.008). Hypertension was less prevalent in the NAG group compared to the control group (OR: 0.35 [95% CI: 0.18; 0.71], p=0.004). Antiviral administration was more frequent in the NAG group compared to the control group (OR: 2.58 [95% CI: 0.88; 7.55], p=0.001). A full list of demographics and clinical characteristics upon admission are in Table 2. Outputs of normality tests are shown in Table 3.

TABLE 2

| Characteristic | NAG (n = 48) | | Control (n = 100) | Comparison | p-value |
|---|---|---|---|---|---|
| Age (years) | 63 (51.50- | | 68 (57.25- | 5.0 | 0.049** |
| | 72.25) [29-88] | | 76.75) [23-95] | (0.0; 10.0) | |
| Sex (male) | 24 | (50%) | 62 (62%) | 0.61 | 0.213 |
| | | | | (0.30; 1.23) | |
| Race | — | | — | 1.17 | >0.999 |
| | | | | (0.38; 3.16) | |
| Hispanic | 43 | (89.6%) | 88 (88%) | — | — |
| White | 5 | (10.4%) | 12 (12%) | — | — |
| Time from | 11.5 (4.75- | | 7 (4- | −3.0 | 0.0008** |
| symptom onset | 11) [1.42-32] | | 15.75) [1-59] | (−6.0; −1.0) | |
| (days) | | | | | |
| Disease | 4 (4- | | 4 (4- | 0.0 | 0.156 |
| severity | 5) [3-5] | | 5) [3-7] | (0.0; 0.0) | |
| Supplemental | 46 | (91.7% | 86 (86%) | 1.79 | 0.425 |
| oxygen | | | | (0.56; 5.22) | |
| Bloodwork | | | | | |
| WBC | 8.65 (6.05- | | 10.25 (6.725- | 1.4 | 0.070 |
| | 11.73) [1.1-20] | | 13.50) [3.3-29.3] | (−0.1; 3.1) | |
| HCT | 39.71 ± 6.02 | | 37.97 ± 7.93 | −1.74 | 0.181 |
| | | | | (−4.29; 0.82) | |
| HGB | 12.69 ± 1.91 | | 12.10 ± 2.61 | −0.59 | 0.175 |
| | | | | (−1.45; 0.27) | |
| Comorbidities | | | | | |
| Asthma | 0 | (0%) | 3 (3%) | <0.01 | 0.551 |
| | | | | (<.01; 2.40) | |
| Atrial fibrillation | 2 | (2.4%) | 7 (7%) | 0.58 | 0.719 |
| | | | | (0.12; 2.80) | |
| Coronary artery | 6 | (12.5%) | 17 (17%) | 0.70 | 0.629 |
| disease | | | | (0.26; 1.95) | |
| Chronic heart | 1 | (2.1%) | 7 (7%) | 0.28 | 0.273 |
| failure | | | | (0.02; 1.63) | |
| COPD | 3 | (6.25%) | 11 (11%) | 0.54 | 0.550 |
| | | | | (0.16; 1.92) | |
| ESRD | 7 | (14%) | 9 (9%) | 1.65 | 0.404 |
| | | | | (0.65; 4.56) | |
| Hyperlipidemia | 13 | (27.1%) | 36 (36%) | 0.66 | 0.352 |
| | | | | (0.32; 1.42) | |
| Hypertension | 22 | (45.8%) | 71 (71%) | 0.35 | 0.0004** |
| | | | | (0.18; 0.71) | |
| Obesity | 27 | (56.25%) | 40 (40%) | 1.93 | 0.078 |
| | | | | (0.97; 3.96) | |
| Smoker | 1 | (2.1%) | 1 (1%) | 2.11 | 0.545 |
| | | | | (0.11; 40.63) | |
| Positive chest x- | 46 | (95.8%) | 87 (87%) | 3.44 | 0.145 |
| ray | | | | (0.82; 15.760 | |
| Dietary | | | | | |
| treatments* | | | | | |
| Antibiotics | 43 | (89.6%) | 94 (94%) | 0.55 | 0.336 |
| | | | | (0.15; 1.71) | |
| Antivirals | 13 | (27.1%) | 6 (6%) | 2.58 | 0.001** |
| | | | | (0.88; 7.55) | |
| Corticosteroids | 43 | (89.6%) | 78 (78%) | 2.43 | 0.112 |
| | | | | (0.87; 6.20) | |
| Convalescent | 2 | (4.2%) | 4 (4%) | 1.04 | >0.999 |
| plasma | | | | (0.19; 4.61) | |

Data are reported as n (%), mean ± standard deviation, or median (interquartile range) [range]. Effect sizes are reported as mean differences (95% CI), Hodges-Lehmann differences (95% CI), or odds ratios (95% CI). P-values were computed using unpaired t-tests (normally distributed data), Mann-Whitney U test (nonparametric data), or Fisher's exact test (dichotomous data).
*Some patients did not receive treatments on day 1. Within the NAG group, one patient received antibiotics on day 5, two patients received antivirals on day 2 and one patient received antivirals on day 3, and three patients received corticosteroids on day 2. All patients in the control group received medications starting on day 1.
**Statistically significant
COPD = Chronic obstructive pulmonary disease;
ESRD = End-stage renal disease;
HCT = Hematocrit;
HGB = Hemoglobin;
WBC = White blood cell count

TABLE 3

| | | | | Passed |
|---|---|---|---|---|
| | | Test | | nor- |
| | | statis- | p- | mality |
| Variable | Test | tics | value | test? |
| Age | Anderson-Darling | 1.364 | 0.002 | No |
| | D'Agostino-Pearson omnibus | 5.758 | 0.056 | |
| | Shapiro-Wilk | 0.974 | 0.007 | |
| | Kolmogorov-Smirnov | 0.117 | <0.001 | |
| Time from | Anderson-Darling | 6.048 | <0.001 | No |
| symptom | D'Agostino-Pearson omnibus | 57.560 | <0.001 | |
| onset to | Shapiro-Wilk | 0.850 | <0.001 | |
| admission | Kolmogorov-Smirnov | 0.151 | <0.001 | |
| WBC | Anderson-Darling | 1.321 | 0.002 | No |
| on | D'Agostino-Pearson omnibus | 25.310 | <0.001 | |
| admission | Shapiro-Wilk | 0.947 | <0.001 | |
| | Kolmogorov-Smirnov | 0.080 | 0.0210 | |
| HGB on | Anderson-Darling | 0.704 | 0.065 | Yes |
| admission | D'Agostino-Pearson omnibus | 3.511 | 0.173 | |
| | Shapiro-Wilk | 0.984 | 0.094 | |
| | Kolmogorov-Smirnov | 0.093 | 0.003 | |
| HCT | Anderson-Darling | 0.766 | 0.045 | Yes |
| on | D'Agostino-Pearson omnibus | 3.743 | 0.154 | |
| admission | Shapiro-Wilk | 0.986 | 0.144 | |
| | Kolmogorov-Smirnov | 0.0734 | 0.050 | |
| Duration of | Anderson-Darling | 8.065 | <0.001 | No |
| oxygen use | D'Agostino-Pearson omnibus | 50.980 | <0.001 | |
| from day 1 | Shapiro-Wilk | 0.814 | <0.001 | |
| | Kolmogorov-Smirnov | 0.208 | <0.001 | |
| ICU LOS | Anderson-Darling | 1.951 | <0.001 | No |
| | D'Agostino-Pearson omnibus | 13.69 | 0.001 | |
| | Shapiro-Wilk | 0.864 | <0.001 | |
| | Kolmogorov-Smirnov | 0.1802 | <0.001 | |
| Hospital | Anderson-Darling | 8.098 | <0.001 | No |
| LOS | D'Agostino-Pearson omnibus | 62.750 | <0.001 | |
| | Shapiro-Wilk | 0.821 | <0.001 | |
| | Kolmogorov-Smirnov | 0.202 | <0.001 | |

Univariate Analysis of Outcomes

Initial analyses of all primary and secondary outcomes are presented in Table 4. The NAG group was significantly less likely to have mortality (12.5% vs. 28.0%, respectively; OR: 0.37 [95% CI 0.15; 0.91], p=0.039) and poor clinical outcome (OR: 0.30 [95% CI 0.12; 0.80], p=0.015) compared to the control group. There were no significant differences for intubation rates, hospital LOS, ICU admission, ICU LOS, duration of oxygen use, or hospice initiation between the control and NAG groups based on univariate analysis (Table 4).

TABLE 4

| | | NAG | | Control | | p- |
|---|---|---|---|---|---|---|
| | Outcome | (n = 48) | | (n = 100) | Comparison | value |
| Pri- | Intubation | 8 | (16.7%) | 25 (25%) | 0.60 | 0.297 |
| mary | | | | | (0.26; 1.48) | |
| | Hospital | 7 (5- | | 7.5 (4- | 0.0 | 0.643 |
| | LOS | 117.5) [2-31] | | 17) [0-59] | (−1.0; 3.0) | |
| | Mortality | 6 | (12.5%) | 28 (28%) | 0.37 | 0.039** |
| | | | | | (0.15; 0.91) | |
| Sec- | ICU | 11 | (22.9%) | 36 (36%) | 0.53 | 0.133 |
| ondary | admission | | | | (0.25; 1.19) | |
| | ICU LOS† | 2.5 (0.75- | | 9.0 (2.25- | 4.0 | 0.092 |
| | | 14.75) | | 21.5) | (−1.0; 12.0) | |
| | Duration of | 7.0 (5- | | 7.0 (3- | 0.0 | 0.834 |
| | oxygen | 12) [2-31] | | 15) [1-53] | (−2.0; 2.0) | |
| | (days)* | | | | | |
| | Hospice | 0 | (0%) | 4 (4%) | 0.00 | 0.149 |
| | initiation‡ | | | | (0.00; 1.52) | |
| | Poor clinical | 6 | (12.5%) | 32 (32%) | 0.30 | 0.015** |
| | outcome | | | | (0.12; 0.80) | |

Data are reported as n (%) or median (IQR) [range]. Effect sizes are reported as Hodges-Lehmann differences (95% CI) or odds ratios (95% CI). P-values were computed using the Mann-Whitney U test or Fisher's exact test.
*The comparison of duration of oxygen use only includes recorded observations from patients that required oxygen support; NAG: n = 46, Control: n = 86.
**Statistically significant
†The comparison of ICU length of stay only includes recorded observations from patients admitted to the ICU; NAG: n = 10, Control: n = 36.
‡The hospice initiation population excludes patients that died; NAG: n = 42, Control: n = 72.
ICU = Intensive care unit;
LOS = Length of stay

Multivariate Analysis of Primary Outcomes

A correlation matrix of all pairs of correlation coefficients between all pairs of covariates was prepared. Following best subset selection for multivariate analysis to maximize adjusted R² values, NAG was shown to be a significant independent predictor of reduced hospital LOS (β: −4.27 [95% CI: −5.67; −2.85], p=<0.001) (Table 5; Multivariate linear regression, regressing background characteristics and comorbidities against hospital length of stay).

TABLE 5

| Variable | β-coefficient | 95% CI | p-value |
|---|---|---|---|
| (intercept) | 0.27 | −4.85; 5.39 | 0.918 |
| NAG (ref = control) | −4.27 | −5.67; −2.87 | <0.001 |
| Time from symptom onset | 0.92 | 0.85; 0.98 | <0.001 |
| HCT | −0.08 | −0.17; 0.02 | 0.108 |
| COPD | −1.44 | −3.47; 0.58 | 0.161 |
| HLD | −0.86 | −2.18; 0.46 | 0.198 |
| Obesity | 0.87 | −0.47; 2.21 | 0.203 |
| ESRD | 2.54 | 0.51; 4.57 | 0.015 |
| Severity | 0.70 | −0.24; 1.64 | 0.145 |
| Antibiotics | 2.44 | −0.10; 4.98 | 0.059 |
| Antivirals | 2.34 | 0.47; 4.21 | 0.015 |
| Steroids | −1.91 | −3.68; −0.14 | 0.034 |
| | RSME | Adjusted R₂ | |
| Summary | 0.903 | 0.870 | <0.001 |

CI = Confidence Interval;
COPD = Chronic obstructive pulmonary disease;
ESRD = End-stage renal disease;
HCT = Hematocrit;
HLD = Hyperlipidemia;
RMSE = Root mean square error;
WBC = White blood cell count
NAG was not shown to be a significant independent predictor of reduced intubation rate (OR: 0.68 [95% CI: 0.19; 2.30], p = 0.541). NAG was not a significant independent predictor of reduced mortality rate (OR: 0.34 [95% CI: 0.09; 1.07], p = 0.081).

Multivariate Analysis of Select Secondary Outcomes

Multivariate analyses were performed for two secondary outcomes of interest: poor clinical outcome and ICU admission. On multiple logistic regression, NAG was shown to be a significant independent predictor of reduced poor clinical outcome rate (OR: 0.30 [95% CI: 0.09; 0.86], p=0.034; see Table 6) and reduced ICU admissions (OR: 0.32 [95% CI: 0.10; 0.96], p=0.049; see Table 7).

TABLE 6

| Variable | OR | 95% CI | p-value |
|---|---|---|---|
| (intercept) | <0.01 | <0.01; 0.004 | <0.001 |
| NAG (ref = control) | 0.30 | 0.09; 0.86 | 0.034 |
| Age | 1.06 | 1.02; 1.10 | 0.003 |
| White (ref = Hispanic) | 2.80 | 0.05; 1.10 | 0.091 |
| Time from symptom onset | 1.05 | 1.01; 1.11 | 0.033 |
| Afib | 4.01 | 0.64; 23.80 | 0.124 |
| CAD | 1.79 | 0.55; 5.79 | 0.324 |
| CHF | 3.21 | 0.46; 20.97 | 0.219 |
| COPD | 2.36 | 0.49; 10.80 | 0.268 |
| ESRD | 5.67 | 1.43; 23.81 | 0.014 |
| Severity | 2.51 | 1.23; 5.55 | 0.016 |
| | ROC | Adjusted pseudo-R² | p-value |
| SUMMARY | 0.856 | 0.415 | <0.001 |

Afib = Atrial fibrillation; CAD = Coronary artery disease; CHF = Congestive heart failure; CI = Confidence Interval; COPD = Chronic obstructive pulmonary disease; ESRD = End-stage renal disease; OR = Odds Ratio; ROC = Area under the receiver operating characteristic curve.

TABLE 7

| Variable | OR | 95% CI | p-value |
|---|---|---|---|
| (intercept) | <0.01 | <0.01; 0.001 | <0.001 |
| NAG (ref = control) | 0.32 | 0.10; 0.96 | 0.049 |
| Age | 1.02 | 0.98; 1.06 | 0.294 |
| Time from symptom onset | 1.10 | 1.05; 1.17 | <0.001 |
| WBC | 1.06 | 0.97; 1.17 | 0.190 |
| HLD | 2.46 | 0.93; 6.68 | 0.070 |
| CXR | 6.11 | 0.87; 64.28 | 0.095 |
| ESRD | 6.83 | 1.71; 30.01 | 0.008 |
| Severity | 3.44 | 1.59; 8.22 | 0.003 |
| Antivirals | 3.20 | 0.75; 13.96 | 0.113 |
| | ROC | Pseudo-R² | p-value |
| SUMMARY | 0.887 | 0.510 | <0.001 |

CI = Confidence Interval;
CXR = Chest x-ray positivity;
ESRD = End-stage renal disease;
HLD = Hyperlipidemia;
OR = Odds Ratio;
ROC = Area under the receiver operating characteristic curve;
WBC = White blood cell count

### Example 8

To assess the use of N-acetyl glucosamine, or a derivative thereof, alone or in combination with one or more additional supplemental agents, such as vitamins or essential minerals, a treating physician will examine a subject (or patient) in a clinical setting for indications of influenza infection, including the use of standard clinical assessments or available diagnostic tests.

The subject (or patient) population will be the general population regardless of age, sex, race, or ethnic origin, and may include subjects (or patients) having pre-existing conditions such as obesity, diabetes, heart disease, autoimmune disorders, or may be otherwise immune compromised.

Upon identification as a subject (or patient) for treatment with N-acetyl glucosamine, or a derivative thereof, the subject (or patient) will be orally administered a composition including at least N-acetyl glucosamine, or a derivative thereof, at a dose (such as 500 mg, 600 mg, 700 mg, 800 mg, 900 mg, or 1000 mg) twice daily (BID dosing) for between 10 and 30 days. The subject (or patient) may be assessed for clinical status by standard clinical assessments or available diagnostic tests at interim periods as determined by the treating physician prior to the end of the treatment cycle. At the determination of the treating physician, the subject (or patient) may be co-administered one or more additional supplemental agents, including vitamin C, folate and/or zinc at standard doses and at an interval to be determined by the treating physician. The results of treatment will be collected and compared to standard of care. The results will be bench-marked against standard of care by metrics including but not limited to recovery rate, death rate, and/or average recovery time.

1. A method of treating a subject having a viral infection, such as influenza, rhinovirus, or betacoronavirus infection, comprising administering a therapeutically effective amount of N-acetyl glucosamine, or a derivative thereof.

2. The method of claim 1, wherein the betacoronavirus infection is SARS-CoV-2 infection.

3. The method of claim 1, wherein the betacoronavirus infection has caused or exacerbated in the subject one or more of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.

4. The method of claim 1, wherein the therapeutically effective amount treats one or more symptoms of the betacoronavirus infection.

5. The method of claim 1, wherein the N-acetyl glucosamine, or a derivative thereof, is administered intravenously, orally, subcutaneously, buccally, transdermally, or nasally.

6. The method of claim 1, wherein the N-acetyl glucosamine, or a derivative thereof, is administered orally.

7. The method of claim 1, wherein the therapeutically effective amount of the N-acetyl glucosamine, or a derivative thereof, is in the range of about 200 mg to about 2100 mg.

8. The method of claim 1, wherein the therapeutically effective amount of the N-acetyl glucosamine, or a derivative thereof, is administered once a day (QD), twice a day (BID), or three times a day (TID).

9. The method of claim 1, wherein the therapeutically effective amount of the N-acetyl glucosamine, or a derivative thereof, is administered twice a day (BID), at a dose of about 300 mg to about 900 mg per dose; or about 600 mg to about 800 mg; or about 700 mg.

10. The method of claim 1, further comprising administration of one or more additional supplement agents.

11. The method according to claim 10, wherein the one or more additional supplement agents is a vitamin or an essential mineral.

12. The method according to claim 10, wherein the one or more additional supplement agents is vitamin A, a B vitamin, vitamin C vitamin D, or zinc.

13. The method of claim 1, wherein the clinical outcome for the patient receiving the treatment is lower rate of ICU admission, reduced hospital length of stay (LOS), lower rate of death, and/or lower rate of hospice initiation.

14. A pharmaceutical composition comprising N-acetyl glucosamine, or a derivative thereof, and optionally a pharmaceutically acceptable carrier or excipient, wherein the N-acetyl glucosamine, or a derivative thereof, is in a therapeutically effective amount for treating a viral infection, such as a betacoronavirus infection.

15. The pharmaceutical composition of claim 14, wherein the N-acetyl glucosamine is in an amount of about 200 mg to about 2100 mg in the composition.

16. The pharmaceutical composition of claim 15, wherein the N-acetyl glucosamine is in an amount of about 300 mg to about 900 mg; or about 600 mg to about 800 mg; or about 700 mg in the composition.

17. The pharmaceutical composition of claim 14, further comprising one or more additional supplement agents.

18. The pharmaceutical composition according to claim 17, wherein the one or more additional supplement agents is a vitamin or an essential mineral.

19. The pharmaceutical composition according to claim 18, wherein the one or more additional supplement agents is vitamin A, a B vitamin, vitamin C. vitamin D, or zinc.

20.-32. (canceled)

33. A compound of the formula 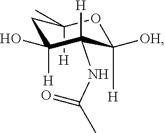
or a pharmaceutically acceptable salt thereof.
